(19) United States
(12) Patent Application Publication
(10) Pub. No.: US 2007/0293808 A1
(43) Pub. Date: Dec. 20, 2007

(54) Renal blood flow augmentation for congestive heart failure treatment

(76) Inventors: Michael S. Williams, Santa Rosa, CA (US); Daniel W. Fifer, Windsor, CA (US)

Correspondence Address: STALLMAN & POLLOCK LLP, 353 SACRAMENTO STREET, SUITE 2200, SAN FRANCISCO, CA, 94111, US

(21) Appl. No.: 11/796,179

(22) Filed: Apr. 27, 2007

Related U.S. Application Data
(60) Provisional application No. 60/795,477, filed on Apr. 27, 2006.

(51) Int. Cl. A61M 27/00

(52) U.S. Cl. 604/9

(57) ABSTRACT

Intravascular devices are delivered to the aorta percutaneously via the femoral artery. The devices are anchored within the vasculature in the region of the renal artery ostia. These embodiments function to increase the flow of blood from the aorta to the renal arteries, thus delivering a higher relative percentage of the blood flowing through the aorta to the kidneys. The elevation in blood low to the kidneys improves the natural removal of excess fluids from the body. In one embodiment, the device is a diverter element positionable upstream of the renal artery ostia. In another embodiment, the device is a flow restrictor positionable downstream of the ostia to cause an elevation is pressure upstream of the ostia.

### PRIORITY

This application claims the benefit of U.S. Provisional Application No. 60/795,477, filed Apr. 27, 2006.

### FIELD OF THE INVENTION

The present invention relates to the field of mechanical and fluid dynamic enhancement to improve the natural removal of excess fluids from the body, such as for treatment of symptoms of congestive heart failure.

### BACKGROUND OF THE INVENTION

Congestive heart failure (CHF) is a chronic, progressive disease in which the myocardium weakens and cannot pump blood efficiently. CHF results in a number of complications, including fluid accumulation in the lungs, hands, ankles, liver and gastrointestinal tract, and other parts of the body. Pulmonary edema is one of the more significant complications of congestive heart failure. As a results of CHF, the condition of the failing heart causes increased pressure to the pulmonary veins. As this pressure increases fluid is pushed into the alveoli of the lungs. The fluid then becomes a barrier to normal oxygen exchange, causing the patient to experience shortness of breath.

Medications to accelerate water excretion from the body via the urine are normally given but these have many side effects including potassium loss and anemia.

### BRIEF DESCRIPTION OF THE DRAWINGS

FIG. 1 is a side elevation view of a first embodiment of a renal blood flow augmentation system;

FIG. 2 is a schematic view showing a cross-sectional side view of a portion of an aorta and renal arteries of a human subject, and further showing the renal blood flow augmentation device of FIG. 1 positioned in the aorta;

FIG. 3 is a schematic view similar to FIG. 2, showing a second embodiment of a renal blood flow augmentation device;

FIG. 4A is a side elevation view of an alternative diversion member that may be used with the augmentation devices of FIGS. 1-3. FIG. 4B is a lateral cross-section view of the member of FIG. 4A as positioned in the aorta.

FIG. 5A is a side elevation view of a second alternative diversion member that may be used with the augmentation devices of FIGS. 1-3. FIG. 5B is a lateral cross-section view of the member of FIG. 5A.

FIG. 6A is a schematic view similar to FIG. 2, showing an alternative renal blood flow augmentation device having a downstream restrictor. FIG. 6B illustrates the downstream restrictor of FIG. 6A in a reduced-diameter configuration for reduced restriction.

FIG. 7 is a schematic view similar to FIG. 6A showing an alternative renal blood flow augmentation device having a downstream restrictor.

FIG. 8 is a schematic view similar to FIG. 6A showing an alternative renal blood flow augmentation device having a pressure-dependent downstream restrictor.

FIG. 9 is a side section view of an alternative configurations of a renal blood flow augmentation devices using a pressure-dependent downstream restrictor system.

### DETAILED DESCRIPTION

The embodiments disclosed herein are intravascular devices delivered to the aorta percutaneously via the femoral artery. The devices are anchored within the vasculature in the region of the renal artery ostia. These embodiments function to increase the flow of blood from the aorta to the renal arteries, thus delivering a higher relative percentage of the blood flowing through the aorta to the kidneys. The elevation in blood flow to the kidneys improves the natural removal of excess fluids from the body.

Although the disclosed devices are illustrated for use in increasing blood flow to both kidneys, alternative designs might be used to increase blood flow to only one of the kidneys.

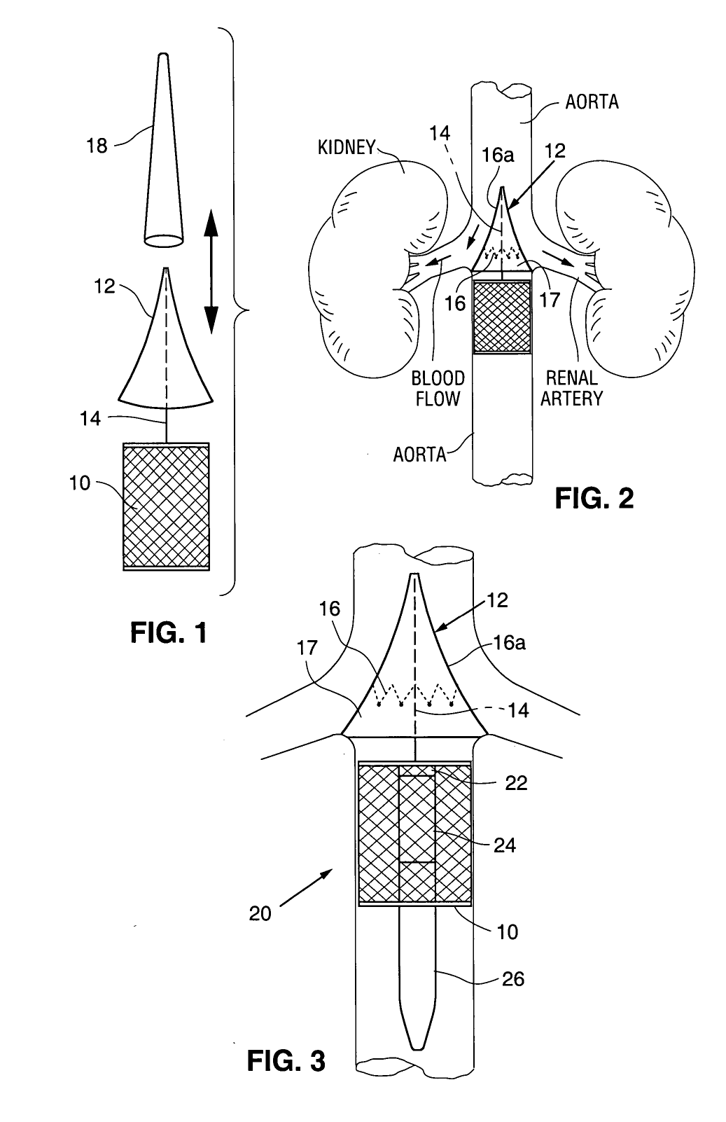

Referring to FIG. 1, a first embodiment of a renal blood flow system generally includes an anchoring device 10, a diversion member 12, and a delivery/retrieval device 20. During use of this embodiment, the diversion member functions to divert blood from the aorta into the renal arteries.

Referring to FIG. 2, the anchoring device 10 includes structural features that allow it to radially engage a vessel wall. For example, a band, mesh or other framework formed of one or more shape memory (e.g. nickel titanium alloy, nitinol, thermally activated shape-memory material, or shape memory polymer) elements or stainless steel, Elgiloy, or MP35N elements may be used. The anchoring device is preferably provided with a smooth polymeric barrier that is both anti-proliferative and anti-thrombogenic and that thereby prevents endothelial growth and thrombus formation on the anchor. Examples of materials for the polymeric barrier include, but are not limited to ePTFE, or other fluoropolymers, silicone, non-woven nylon, or biomimetic materials. Other suitable anchors might be similar to anchors of the type used to anchor vena cava filters within the vasculature.

The anchoring device 10 is preferably compressible into a sheath or similar deployment device for passage through the vasculature to the aorta, and is then releasable from the deployment device and expandable into engagement with the surrounding walls of the aorta. Applicant's co-pending U.S. application Ser. No. 10/453,971 entitled DEVICE AND METHOD FOR RETAINING A MEDICAL DEVICE WITHIN A VESSEL filed Jun. 4, 2003; Ser. No. 10/862,113, entitled INTRAVASCULAR ELECTROPHYSIOLOGICAL SYSTEM AND METHODS, filed Jun. 4, 2004; Ser. No. 10/977,060, entitled METHOD AND APPARATUS FOR RETAINING MEDICAL IMPLANTS WITHIN BODY VESSELS, filed Oct. 29, 2004, each of which is incorporated herein by reference, disclose various anchor configurations, deployment methods, and methods for coupling implants to intravascular anchors.

Diversion member 12 is delivered and attached to the anchor by a tether 14, although it may alternatively be deployed pre-attached to or integral with the anchoring device 10. The diversion member may comprise of a first collapsed configuration which allows delivery of the implant and ultimately retrieval of the implant. For example, the diversion member (with the anchor or separate from the anchor) may be deployed from and retrieved into a delivery/retrieval capture cone 18 (FIG. 1) or a sheath manipulatable from outside the body to deploy/capture the diversion member 12. Upon deployment, the diversion member may assume a conical shape, with the largest diameter of the cone generally positioned against the aortic wall just inferior to the renal arteries. In other words, once positioned, the diversion member is expanded to a shape that will divert blood from the aorta to the renal arteries as illustrated by arrows in FIG. 2.

Diversion member 12 may be formed of any of a variety of materials or combinations of materials that will allow it to be compressed for deployment and expanded within the blood vessel. For example, the member may be constructed of a nitinol frame 16, 16a shape set into the desired expanded conical shape and covered with flexible, compliant polymer, mesh webbing or a porous or impermeable cover 17 or membrane. Alternatively, the member may be formed of metallic or polymeric braid, mesh or laser cut nitinol tubing which may or may not be coated or impregnated with polymeric material or other coverings or membranes. As another example, the diversion member may be a polymeric piece formed by molding or other suitable processes.

The diversion member may be self-activated (e.g. through the use of self-expandable shape memory elements 16 and/or a shape memory frame 16a) or it may be controlled with a motor, balloon, worm screw, umbrella sliding lock etc. The diversion member may remain in the expanded position once deployed, or it may include electronics and associated features that allow it to be activated on-demand using sensor biofeedback or telemetric controls.

In one method for using the illustrated system, the anchoring device 10 is placed 1-10+ cm below (i.e. downstream of) the renal arteries in the descending aorta. The diversion member is coupled to the anchoring device downstream of the renal arteries and extends superiorly beyond the ostia of the renal arteries. In its deployed state it deflects all or some of the blood flow directly into the renal arteries. It is preferred to maintain an adequate amount of blood supply to the peripheral vessels downstream of the diversion member. One example of flow distribution is ⅔ diversion to the renal vasculature and ⅓ diversion or trickle to the peripheral vasculature.

In a slightly modified embodiment shown in FIG. 3, an intravascular drug delivery device 20 including a pump 22, power supply 24, and drug reservoir 26 may be coupled to the anchor 10 and used to deliver agents into the blood stream. Various embodiments of intravascular drug delivery systems are disclosed in Applicant's co-pending U.S. patent application Ser. No. 11/055,540, entitled INTRAVASCULAR DELIVERY SYSTEM FOR THERAPEUTIC AGENTS, which is incorporated herein by reference.

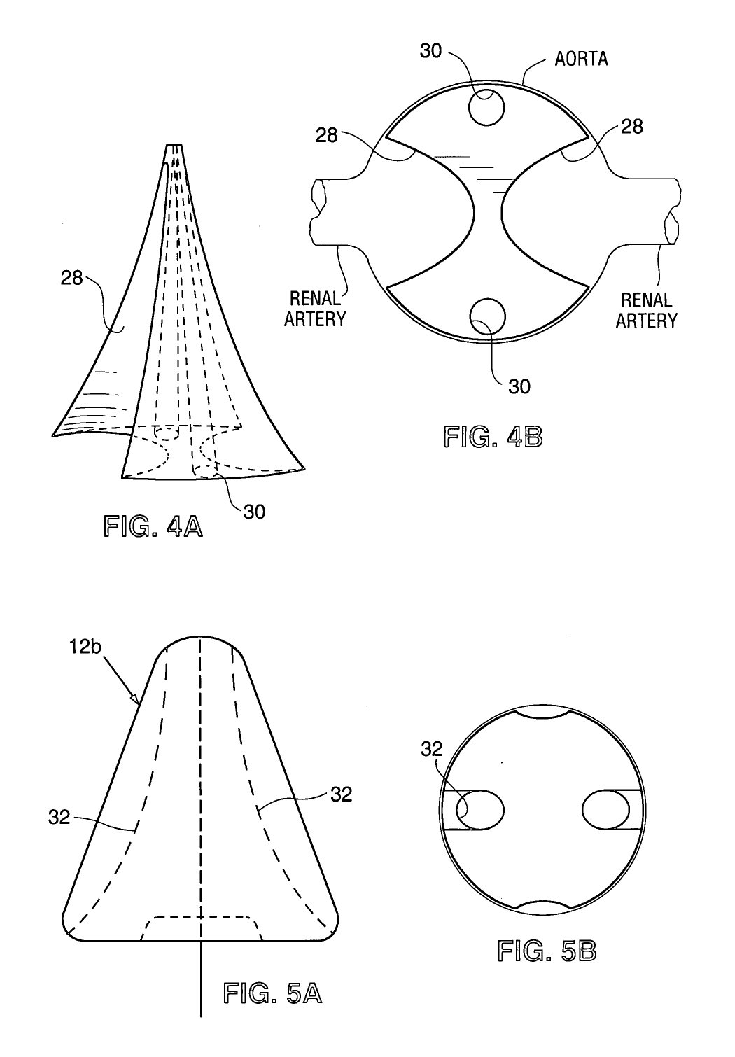

Referring to FIG. 4A, in an alternative diversion member 12a, the conical shape of the diversion member may include concave walls 28 positionable as shown in FIG. 4B to form channels in communication with the renal arteries, to minimize hemolysis while diverting blood into the renal arteries. It may also have side ports 30 to allow some blood to trickle past the diversion member in order to maintain peripheral blood supply.

In addition to flow diversion, the application of a venturi type device which employs the Bernoulli principle may be incorporated into the diversion member design. According to the Bernoulli principle, as a fluid passes through a pipe that narrows or widens the velocity and pressure of the fluid vary. As the pipe or tube narrows, the fluid flows more quickly and simultaneously the pressure drops. When features making use of the Bernoulli effect are employed in the diversion member, they will increase the velocity of blood into the renal arteries which may enhance renal function. In one implementation of a device using the Bernoulli effect, the concave walls 28 of the FIG. 4A may be shaped such that the channels formed between the walls 28 and the wall of the aorta narrow near the renal arterial ostia.

According to an alternative deflection device 12b shown in FIGS. 5A and 5B, deflection/diversion of blood may also be achieved using a balloon device that deploys into its shape upon inflation. The balloon device may be provided with or without channels having venturi ports 32 and/or trickle ports of the type described with respect to FIG. 4A.

A venturi type device might alternatively be positioned over the renal artery ostia, to accelerate flow of blood from the aorta into the ostia.

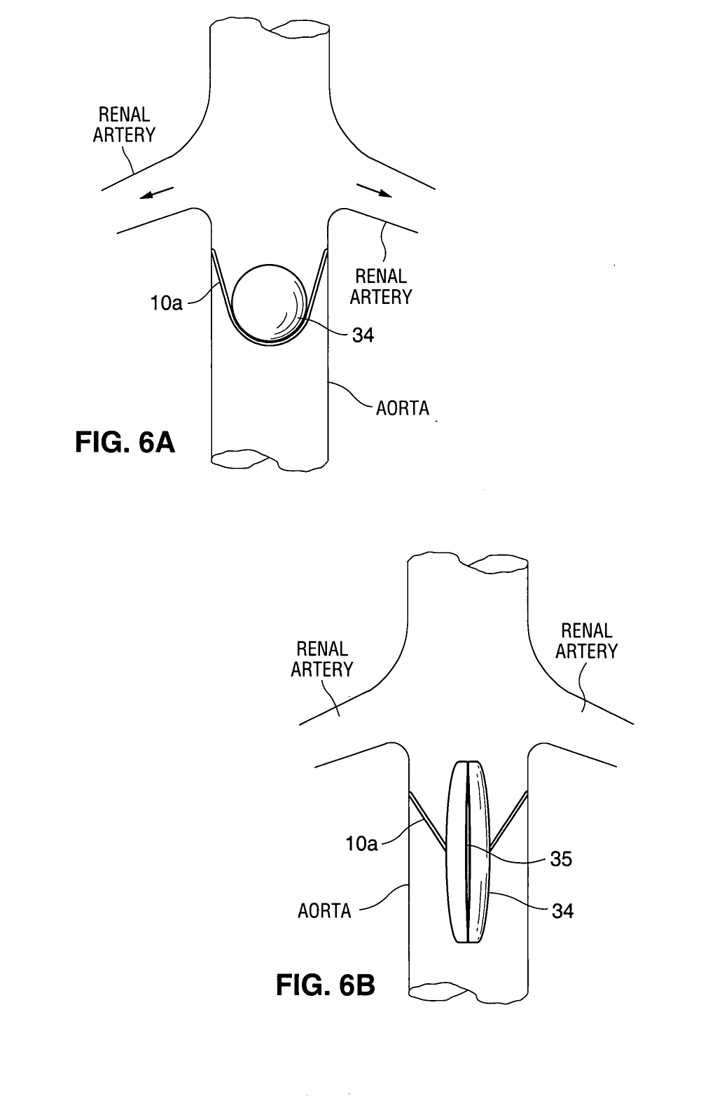

Additional embodiments shown in FIGS. 6A through 9 augment renal blood flow using a restrictor device that restricts blood flow downstream of the renal arteries, thus creating a higher arterial pressure at the renal artery ostia, which results in diversion of a greater percentage of the blood to the kidneys.

In one such embodiment shown in FIG. 6A, restrictor 34 is in the form of an obstructive element suspended by an anchor 10a within the aorta, downstream of the renal arteries. The illustrated anchor is a spider-type anchor of the kind used for anchoring vena cava filters, however other forms of anchors may be used.

The restrictor 34 is shown as a spherical element, but it can be of any size and shape that will cause a sufficient increase in arterial pressure at the renal artery ostia to produce the desired increase in blood flow into the ostia. As illustrated in FIG. 6B, the amount of restriction caused by the restrictor 34 may be altered by changing the lateral dimensions of the restrictor 34. Such changes may be feedback-based such that an active member 36 within the restrictor 34 is activated upon detection of certain mechanical, chemical, physiological, hormonal etc. conditions within the body indicating the need for an increase or decrease in the amount by which blood is diverted in the renal arteries. Feedback may be wirelessly communicated from internal or extracorporeal sensor devices to electronic elements within the restrictor 34

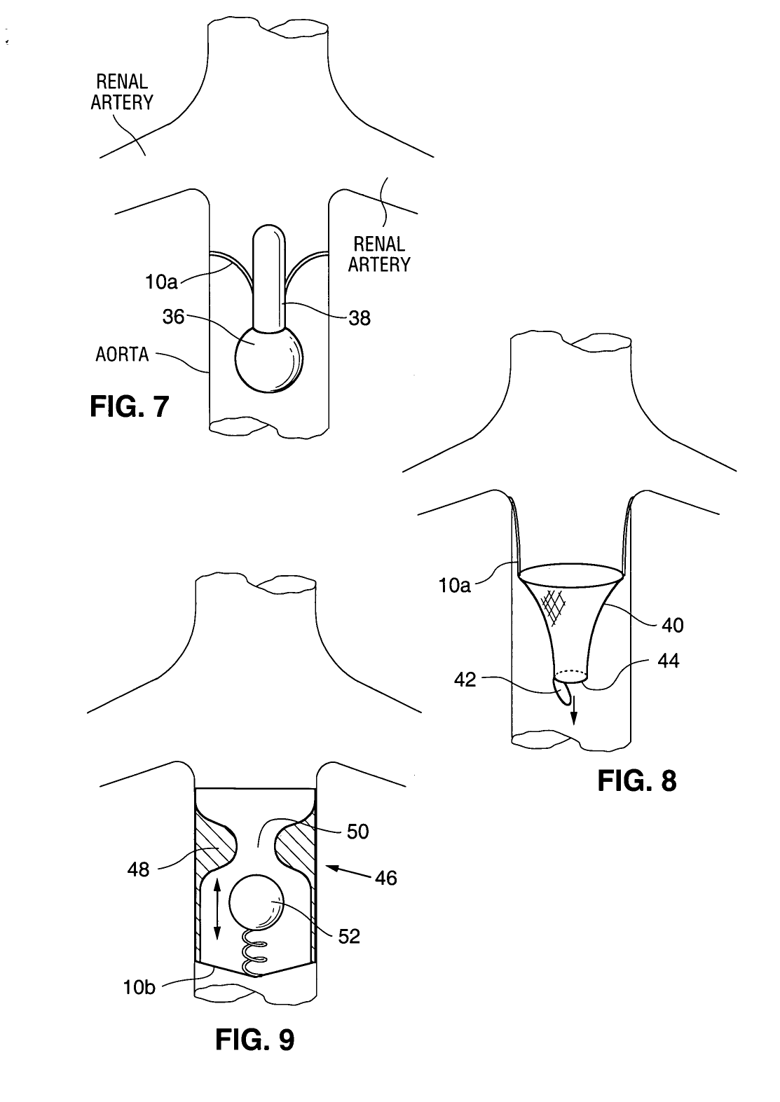

Referring to FIG. 7, the restrictor 34 may be an inflatable bladder 36, thus allowing dimensional changes to be made using a source of inflation medium (e.g. saline, CO2, etc.) delivered to the bladder 36 using a pump 38. The pump 38 may be a two-way pump allowing inflation medium to be injected into the bladder 36 to increase the amount of restriction, and to be drawn back into the reservoir to decrease restriction. Alternatively, the device might include a vent that allows inflation medium to be slowly vented into the bloodstream to reduce the amount of restriction.

In another embodiment shown in FIG. 8, the restrictor is responsive to the cyclical pressure changes within the artery to increase/decrease the amount of restriction. In this type of embodiment, the restrictor may be a funnel 40 having a flap valve 42 spring biased in a closed position covering the distal opening 44 of the funnel 40. When relatively lower pressures are present in the aorta, the flap valve 42 will remain closed, thus diverting a larger amount of blood to the renal arteries. However, when the pressure cycles to the higher-pressure end of the pressure cycle, the flap valve 42 will pivot open, thus diverting less blood to the renal arteries. As a result of this device, the mean arterial pressure at the renal arteries will be higher than would be experienced without the device.

FIG. 9 shows an alternative to the FIG. 8 embodiment, in which the restrictor 46 is in the form of a ball valve that may be mounted within a stent-type anchor 10b or supported by any other suitable anchor. In the FIG. 9 embodiment, the ball valve includes wall 48 extending radially inwardly to form an orifice 50, and a spring mounted ball 52 biased to stop or limit flow through the orifice 50 in the presence of low pressure in the aorta so as to cause a greater volume of blood to flow to the kidneys. In the presence of high pressure in the aorta, the ball is deflected away from the orifice. The ball valve of FIG. 9 may be replaced by a flap valve or any other type of valve that will respond to pressure changes.

In other alternate designs, the anchor may be positioned in the renal artery.

The system is preferably provided as a kit including instructions for use informing the user of the implantation steps described herein, including the steps of percutaneously introducing the implant into the aorta, and anchoring the implant within the vasculature to cause the implant to increase the amount of blood flowing from the aorta into the renal arteries.

While certain embodiments have been described above, it should be understood that these embodiments are presented by way of example, and not limitation. While these systems provide convenient embodiments for carrying out this function, there are many other instruments or systems varying in form or detail that may alternatively be used within the scope of the present invention. This is especially true in light of technology and terms within the relevant art(s) that may be later developed. Moreover, the disclosed embodiments may be combined with one another in varying ways to produce additional embodiments.

Any and all patents, patent applications and printed publications referred to above are incorporated by reference, including those relied upon for purposes of priority.

We claim:

1. A method for augmenting blood flow to a renal artery extending from an aorta, comprising the step of:
anchoring a device within vasculature of a patient in a region of an ostium of the renal artery, the device having a wall positioned to increase flow of blood through the ostium.

2. The method of claim 1, wherein the method includes positioning the wall to divert blood from the aorta through the ostium.

3. The method of claim 1 wherein the method includes anchoring the implant in the aorta.

4. The method of claim 1, wherein the method includes positioning the implant upstream of the ostium.

5. The method of claim 1, wherein the wall has a first portion positioned upstream of the ostium and a second portion positioned downstream of the first portion.

6. The method of claim 5 wherein the wall flares outwardly from the first portion to the second portion.

7. The method of claim 1, wherein the method includes positioning the implant downstream of the ostium, and wherein the wall restricts flow of blood past the implant.

8. The method of claim 7, including the step of altering the amount of restriction created by the implant.

9. The method of claim 8, wherein the implant is moveable between expanded and contracted positions to increase and decrease the amount of restriction.

10. The method of claim 8, wherein the implant includes an orifice having a valve, and wherein the method includes moving the valve between opened and closed positions to alter the amount of restriction.

11. The method of claim 10, wherein the method includes opening the valve in response to a first pressure in the aorta, and closing the valve in response to a second pressure in the aorta, the first pressure higher than the second pressure.

12. The method of claim 1, wherein the implant defines a channel including a narrowing in the channel, and wherein the method includes accelerating blood flow through the channel.

13. The method of claim 1, wherein the method includes percutaneously introducing the implant into the vasculature.

14. The method of claim 1, wherein the method is for increasing blood flow from the aorta to a pair of renal arteries each having an ostia, and wherein the wall is positioned to increase blood flow from the aorta through the ostia.

15. A system for increasing blood flow from an aorta to a renal artery, comprising:
an element positionable in an aorta in the region of a renal artery ostium, the element including a wall oriented to increase flow of blood from the aorta to the renal artery; and
an anchor coupled to the element, the anchor expandable into contact with a wall of the aorta to anchor the element therein.

16. The system according to claim 15, wherein the implant is a flow diverter, wherein at least a portion of the wall is positionable upstream of the ostium to divert flow of blood from the aorta to the renal artery.

17. The system according to claim 15, wherein the wall has a first portion positionable upstream of the ostium, a second portion positionable downsteam of the first portion, wherein the wall flares outwardly from the first portion to the second portion.

18. The system according to claim 15, wherein the implant is a flow restrictor, wherein at least a portion of the wall is positioned downstream of the ostium to restrict flow of blood past the flow restrictor.

19. The system according to claim 18, wherein the restrictor is expandable and contractable to increase and decrease an amount of restriction.

20. The system according to claim 19, wherein the implant includes an orifice having a valve, and wherein the method includes moving the valve between opened and closed positions to alter the amount of restriction.

21. The system according to claim 15, wherein the implant includes a venturi.
